(19) United States
(12) Patent Application Publication
(10) Pub. No.: US 2005/0216020 A1
(43) Pub. Date: Sep. 29, 2005

(54) Assembly for use in orthopaedic surgery

(76) Inventor: Marcus Orton, Boston Spa (GB)

Correspondence Address: Paul J Maginot;Maginot Moore & Beck, Bank One Center Tower, 111 Monument Circle Suite 3000, Indianapolis, IN, 46204-5115, US

(21) Appl. No.: 10/513,563

(22) PCT Filed: May 6, 2003
(86) PCT No.: PCT/GB03/01901

§ 371 Date: May 25, 2005

(30) Foreign Application Priority Data
May 7, 2002 (GB) 02103638

(51) Int. Cl. A61B 17/32

(52) U.S. Cl. 606/80

(57) ABSTRACT

An assembly for use in orthopaedic surgery comprises a component which is to be positioned within a body cavity to engage a bone the component comprises a hollow shell which is open on one side to allow access to its interior and has a bar extending across it. The assembly includes a manipulator having a clasp for engaging the bar so as to fasten the component to the manipulator, in which the clasp allows rotation of the bar so that the angular orientation of the component relative to the manipulator can be changed.

This invention relates to an assembly for use in orthopaedic surgery, which comprises a hollow shell component having a bar extending across it, and a manipulator having a clasp for engaging the bar so as to fasten the component to the manipulator.

Hollow shell components have uses in orthopaedic surgery such as shaping bone tissue, for example to receive a component of an orthopaedic joint prosthesis, and as orthopaedic joint prosthesis components. An instrument having a hollow shell configuration has to be manipulated when used to shape a bone. For example, when the instrument is a cutting tool with cutting teeth on its external surface, it can be rotated about an axis of symmetry (for example when the external surface defines part of a sphere) to cause the bone tissue to be cut. A component of a joint prosthesis has to be manipulated to ensure that it is aligned properly with the prepared surface of the bone.

It is known to provide a hollow shell component with a bar which extends across it which can be engaged by a manipulator with an appropriate clasp. In the case of an instrument, the bar does not need to be detached from the shell, and can be bonded to the shell or formed integrally with it, for example by casting. In the case of a component of a joint prosthesis, the bar can be attached to the shell component by means of appropriate formations, for example which engage a lip on the component.

It is desirable to minimise the size of the incision that is necessary during surgery, for example to minimise blood loss and damage to soft tissue, as well as for aesthetic reasons. When performing surgery on a patient's hip joint, especially when implanting an acetabular cup prosthesis, it is generally the case that the incision has to be capable of accommodating the cup prosthesis itself when directed towards the acetabulum, aligned appropriately relative to the relevant axis.

The present invention provides an assembly for use in orthopaedic surgery which comprises a shell component and a manipulator, in which a clasp on the manipulator engages a bar on the shaft, so as to allow rotation of the bar within the clasp.

Accordingly, in one aspect, the invention provides an assembly for use in orthopaedic surgery, which comprises a component which is to be positioned within a body cavity to engage a bone, the component comprising a hollow shell which is open on one side to allow access to its interior and which has a bar extending across it, and a manipulator having a clasp for engaging the bar so as to fasten the component to the manipulator, in which the clasp allows rotation of the bar so that the angular orientation of the component relative to the manipulator can change.

The assembly of the present invention has the advantage that, for a given size of shell component and a given manipulator, the component can be delivered to the relevant bone through a smaller incision than might be necessary using known assemblies, by changing the angular orientation of the component relative to the manipulator.

Preferably, the bar which has a generally rounded cross-section. Preferably, the clasp has a generally rounded recess in which the bar can be received. These features can facilitate rotation of the bar within the clasp.

Preferably, the clasp comprises a recess which is shaped to receive the bar, and a locking part which can be moved between two positions in which (a) the bar is prevented from moving out of the recess, and (b) the bar can be moved out of the recess, respectively. For example, the recess can be approximately C-shaped, so that the bar is received in the recess by being slid transversely. A retractable pin can then close the recess, which allows the bar to be moved out of the recess when retracted. The pin can be profiled so that it can be displaced by the bar when force is applied to the bar to force it into the recess.

Alternatively, cooperating formations on the clasp and the head can engage when the bar is received in the recess to prevent the bar from being withdrawn from the recess. For example, a protruding formation such as a ridge or pin can be received in an appropriate recess formation (which might be a groove when it is intended to receive a protruding formation in the form of a ridge).

Preferably, the clasp includes at least two recesses. For example, when the bar extends across the shell, the clasp can include two recesses to engage the bar on opposite sides of the centre. Preferably, each recess has a respective locking part, for example comprising a protruding formation and a recess formation.

Preferably the movable locking parts of the clasp which cooperate with respective recesses for the bar can be moved together between the locked and released positions. For example each of the locking parts can be provided on a sliding collar.

Preferably, the clasp has a lock which can engage the bar to restrict movement of the bar relative to the clasp. Preferably, the lock can be moved between a first position in which the bar can move relative to the manipulator and a second position in which movement of the bar is restricted. The movement which is restricted by the lock can involve (a) rotation of the component about the axis defined by the bar when it is received within the clamp, or (b) movement of the component relative to the manipulator during assembly of the component on the manipulator.

For example, the bar can have at least one aperture in it, and the clasp can include a retractable pin which can be received in the aperture in the bar to lock the bar against rotation relative to the clasp. The bar can have a flat on one side (or more than one flat, for example two flats on opposite sides) and the lock can comprise a C-shaped collar which a flat side, which can only fit on to the bar when the flat on the bar and the flat side on the collar are aligned. Preferably, the lock is biassed towards the position in which it restricts rotation of the bar. The lock (for example the pin or the C-shaped collar) can be mounted on a collar which can slide relative to the clasp. The lock can be provided on the same collar as locking parts by which the bar is retained within a recess of the clasp.

A preferred lock comprises at least one recess which is provided in one of the component and the manipulator, and at least one ridge which is provided in the other of the component and the manipulator. When the relative rotational positions of the component and the manipulator are such that the ridges are aligned with the recesses the ridges can be received in the recesses. Further rotation of the component relative to the manipulator is not possible while the ridges are received in the grooves.

Preferably, the assembly includes an actuator for causing the angular orientation of the component relative to the manipulator to change. The actuator can comprise an actuator part on the manipulator which can be moved relative to the clasp. The actuator can include a hook which is provided on one of the component and the actuator part, and a recess which is provided in the other of the component and the actuator part. The angular orientation of the component can be changed by moving the actuator part relative to the clasp, which causes the component to move by virtue of the hook being received in the recess.

The actuator part can comprise a sleeve which can be slid relative to a clasp head, by which the bar is engaged by the manipulator. The sleeve can provide a recess which is defined by a hook, which can receive a hook on the component beneath it. When the hook on the component is received in the recess on the sleeve, sliding the sleeve relative to the clasp head can cause the angular orientation of the component relative to the manipulator to change, by rotation of the component about the axis defined by the bar, engaged within the clasp on the manipulator.

The sleeve can carry formations which engage corresponding formations on the component, to lock the component in a desired orientation. For example, ridges or recesses on the manipulator, which fit into corresponding recesses or ridges on the component, can be provided on the sleeve.

Preferably, the shell has a cut out portion towards the open side in a region of its wall that is located approximately opposite to the midpoint of the bar. This can be of particular advantage when the bar can rotate relative to the clasp because it can reduce interference of the manipulator with rotation of the component and therefore allow rotation of the component through a larger angle.

The external surface of the shell can be symmetrical about an axis of rotation. For example, the external surface can define a part of a sphere. This might be the case when the assembly is for use in connection with a joint prosthesis in which one component articulates against the other component in the manner of a ball which is received in a cup. While the configuration of the external surface can preferably be symmetrical about an axis of rotation, the component need not be symmetrical in this way. For example, the shell can have one or more cut-out portions. The wall thickness of the shell can vary from one region to another. The shell can have features on its outer surface according to its intended purpose: for example, the component might be a cutting tool, in which case it can have cutting teeth on its outer surface, and the arrangement of the teeth on the surface need not necessarily be symmetrical about the axis of symmetry.

When the component is a cutting tool, the use of a rotationally symmetrical component has the advantage that the tool can be rotated about the axis to cut the patient's bone tissue. When the external surface of the component defines a part of a sphere, the tool can be rotated about its axis to cut the patient's bone tissue, while at the same time the orientation of the axis relative to the bone is changed. This can be important to achieve satisfactory cutting of the patient's bone in preparation for implantation of a joint prosthesis component.

Preferably, the bar to which the clasp fastens intersects the axis of symmetry.

Preferably, the portion of the bar which is engaged by the clasp is located within the shell so that, when the component is fastened to the manipulator, the clasp is located at least partially within the shell.

Preferably, the ratio of the depth of the centre of the bar within the shell measured from the open side of the shell, to the length of the axis measured from the open side of the shell to the external surface of the shell opposite the open side, is at least about 0.2. When the bar intersects the axis of symmetry of the component, the depth of the bar is measured from the open side of the shell to the point where the centre of the bar intersects the axis of symmetry. When the bar does not intersect the axis of symmetry, the depth of the bar is measured along the axis, to the point at which the centre of the bar is closest to the axis. Preferably, the value of the said ratio is at least about 0.4, more preferably at least about 0.5.

The bar can be straight (when viewed along a line perpendicular to the axis of the shell and to the bar), in which case, it will be fastened to the internal wall of the shell at its ends, at the same depth as point at which the clasp engages the bar. The bar can be cranked, so that the depth of the ends of the bar within the shell need not be the same as the depth of the point at which the clasp engages the bar. For example, the ends of the bar can be located closer to the open side of the shell than the point at which the clasp engages the bar, for example with the ends of the bar fastened to the shell at the open side. Preferably, the bar is approximately straight in the region thereof in which it is engaged by the clasp. The ends of the bar can be located further from the open side of the shell than the point at which the clasp engages the bar; for example the ends of the bar can be fastened to the internal surface of the shell close to the pole of the shell. The bar can also be mounted on a fixture which is fastened to the internal wall of the shell at or close to the pole: for example, the fixture can comprise a length of a tube, and the bar extends across the tube.

The bar is preferably straight when viewed along the axis of the shell (which is axis of symmetry when the shell is rotationally symmetrical). A bar can be considered as consisting of a plurality of limbs extending from the polar axis of the component: for example, a single bar which extends across the component from one side to the opposite side consists of two limbs. The bar can include more than two limbs or there can be more than one bar. For example, there can be two bars which are fastened together at about the shell axis, so that there are four limbs extending radially from the axis. The bars can then be perpendicular to one another at the point at which they are fastened together. Other arrangements are envisaged, for example in which the bar is provided by three limbs which are joined together so that the angle between any two of the limbs is about 120°. The limbs can be joined together at or close to the axis of the component. They might however not extend to the axis of the component and be joined together by means of a ring which encircles the axis.

Preferably, the clasp comprises a recess which is shaped to receive the bar, and a locking part which can be moved between two positions in which (a) the bar is prevented from moving out of the recess, and (b) the bar can be moved out of the recess, respectively. Preferably, the clamp defines at least one recess which can present a transverse opening which allows the bar to be positioned in the recess by sliding it transversely into the recess. The clamp can provide a plurality of recesses, corresponding to each limb of the bar or bars: for example, when there is one bar which extends across the component.

For example, the recess can be approximately C-shaped, so that the bar is received in the recess by being slid transversely relative to the axis of the component. The transverse sliding of the bar can involve relative translocation of the whole component relative to the clasp, or relative rotation (especially without translocation) between the component and the clasp in a plane which is transverse to the axis of the component, or both.

A retractable locking formation, such as a pin or a ridge, can close a recess, in which retraction of the pin allows the bar to be moved out of the recess. A locking formation can be profiled so that it can be displaced by the component when force is applied to the component to connect it to the manipulator.

Preferably, the clasp includes at least two recesses. For example, when the bar extends across the shell, the clasp can include two recesses to engage the bar on opposite sides of the centre. When the bar has three limbs, the clasp can present three recesses. The recesses can be arranged so that the clasp is rotationally symmetrical, facilitating engagement of the component with the clasp by relative rotation. Each recess can have a respective locking part.

Preferably the movable locking parts of the clasp which cooperate with the component to lock it, especially with respective recesses for the bar, can be moved together between the locked and released positions. For example each of the locking parts can be provided on a sliding collar.

Preferably, the manipulator includes a tube portion and a shaft which can rotate within the tube portion, the clasp being fastened to the shaft so that the component can be rotated relative to the tube portion. The assembly can include a handle which is provided by or fastened to the tube portion. The shaft can be driven by a rotary drive unit, especially when the component is a cutting tool.

Embodiments of the present invention will now be described by way of example with reference to the accompanying drawings in which:

FIG. 1 is an isometric view of a shell component of an assembly according to the invention.

FIG. 2 is an exploded isometric view of an assembly according to the invention.

FIG. 3 is an exploded side view of the assembly shown in FIG. 2.

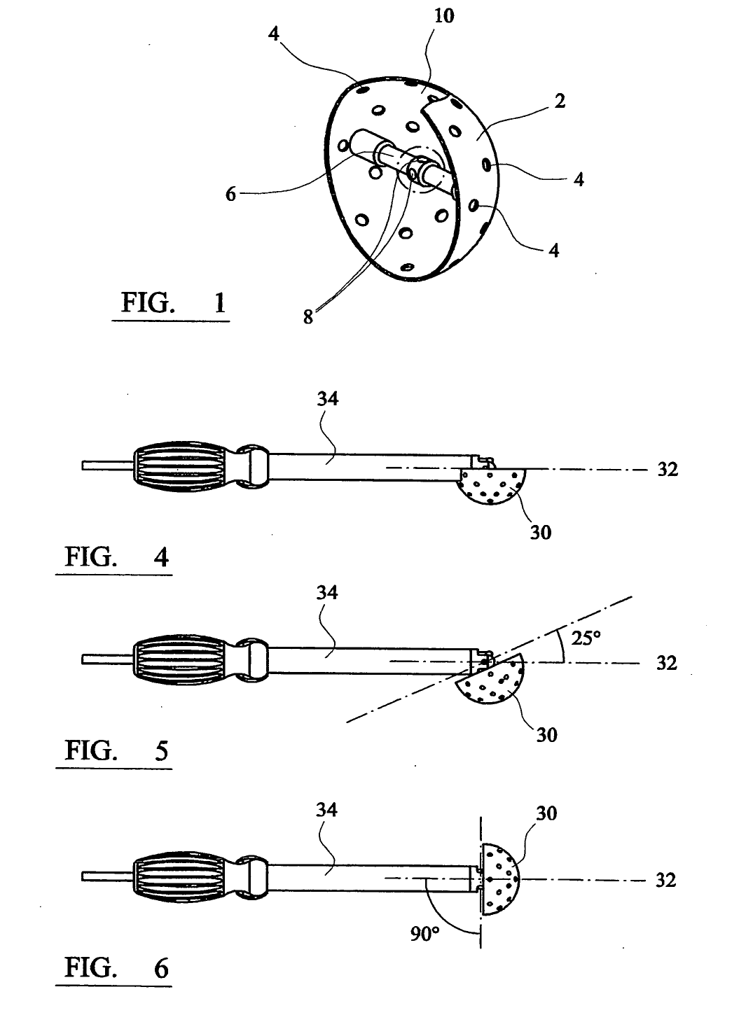

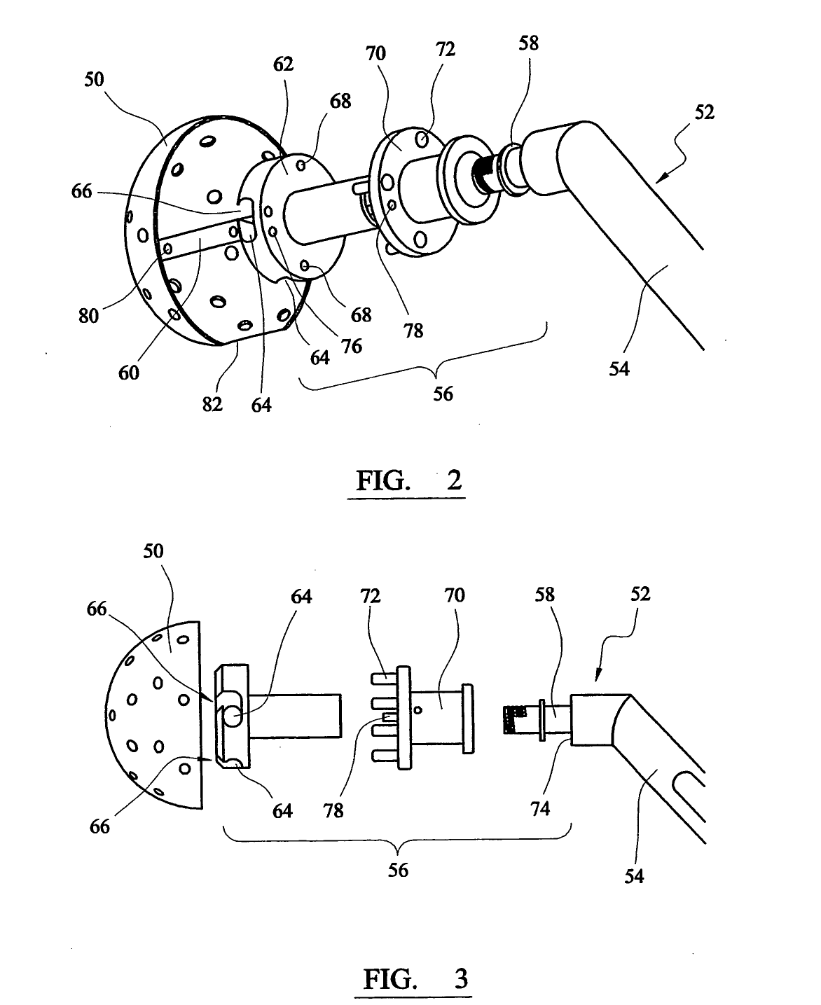

FIGS. 4 to 6 are side views of an assembly according to the invention with the shell component in different angular orientations relative to the manipulator.

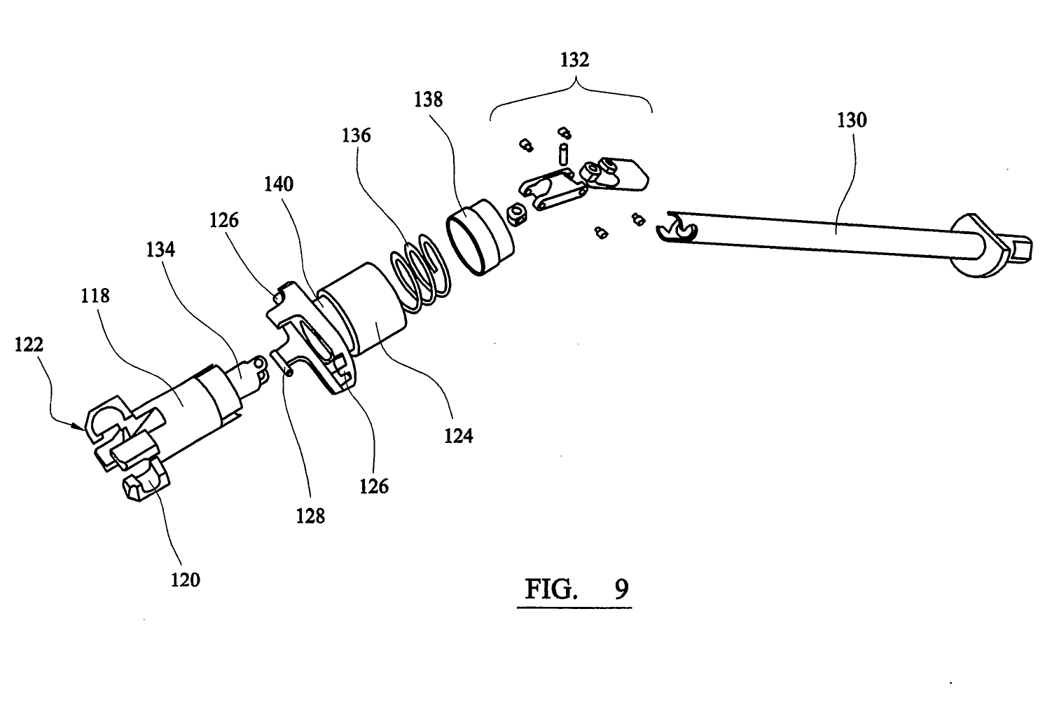

FIG. 7 is an isometric view from below of a preferred embodiment of reamer component.

FIG. 8a is an isometric view of the clasp assembly of a manipulator which can be used with the reamer component shown in FIG. 7.

FIG. 8b is another isometric view of the clasp assembly shown in FIG. 8a, viewed along the arrow “VIII”.

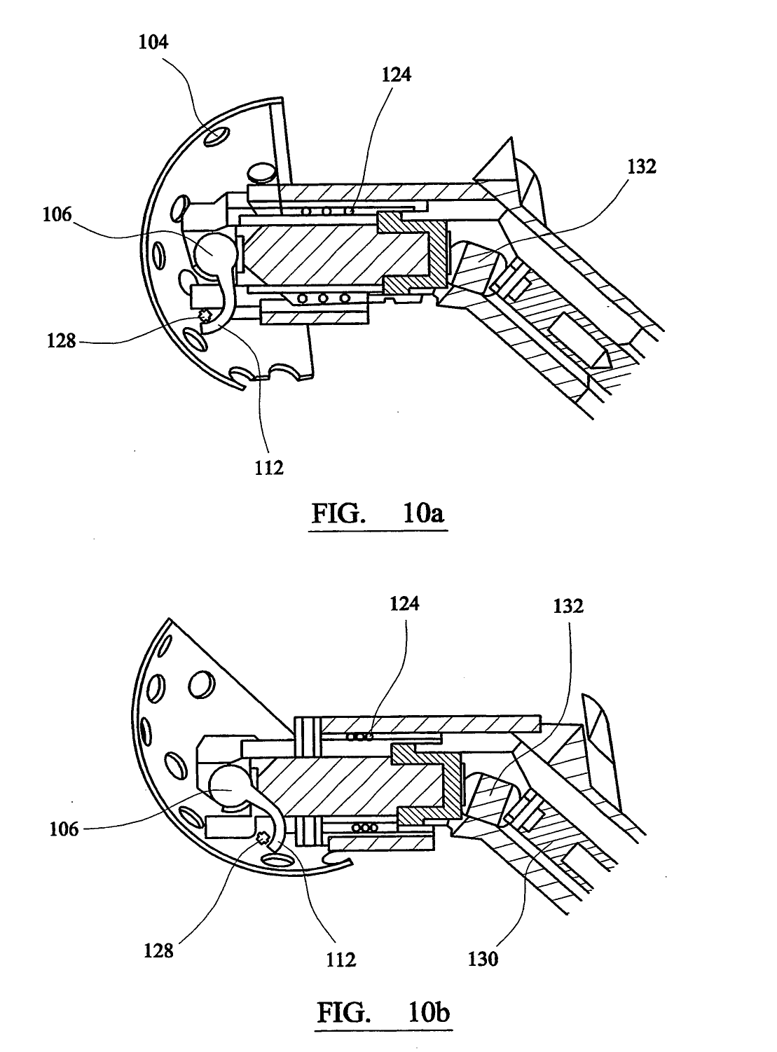

FIG. 9 is an exploded view of the manipulator shown in FIGS. 7 and 8.

FIGS. 10a and 10b are sectional elevations from one side of the reamer component and manipulator shown in FIGS. 7 and 8 respectively, in two angular orientations.

Referring to the drawings, FIG. 1 shows a reamer component of an instrument set which can be used in the implantation of the acetabular cup component of a hip joint prosthesis. The component comprises a hollow shell 2 formed from a suitable metallic material (for example a stainless steel) which has raised cutting teeth 4 arranged on its outer surface. The external surface of the shell defines a part of a sphere and the open side of the shell is at or slightly above about the equatorial plane of that sphere, so that the shell is symmetrical about the polar axis of the shell. Each of the cutting teeth is directed so as to cut the surface of a bone when rotated about the axis of symmetry. The shell is provided by a thin sheet of the metallic material, and can be manufactured by forming a sheet, or by other techniques such as casting and appropriate subsequent finishing.

The reamer component shown in FIG. 1 has a bar 6 within it. The bar is fastened at its ends to the internal surface of the shell, for example by welding. The bar is straight, when viewed along the axis of the sphere, and also when viewed from one side.

The bar 6 in the reamer head has a plurality of lock holes 8 within it at its centre point. The shell has a cut out portion 10 in its outer wall.

FIGS. 2 and 3 show an assembly according to the invention which comprises a reamer head 50 and a manipulator 52. The manipulator comprises a proximal portion 54 with which the assembly can be held. and a distal portion 56. The angle between the proximal and distal portions is about 135°. Each of the proximal and distal portions is hollow and a drive shaft 58 extends through them, via a universal joint at the point where the portions are joined.

The reamer head has a single bar 60 within it, extending between the opposite internal surfaces of the head.

The manipulator includes a clasp head 62 which has four recesses 64 in it. Each of the recesses has an opening 66 on the face of the clasp head which is directed into the shell of the reamer head. The clasp head has a hole 68 extending through it associated with each of the recesses 64. A collar 70 which can slide relative to the clasp head along the distal portion of the manipulator has four closure pins 72 on it, directed along the axis of the distal portion, so that they can slide into and out of the holes 68 in the clasp head. The collar is resiliently biassed towards the clasp head by means of a spring acting against a biassing face 74.

One of the recesses 64 in the clasp head also has a lock pin hole 76 associated with it, extending through the clasp head into the opening. The collar 70 has a lock pin 78 on it, again directed along the axis of the distal portion, so that it can slide into and out of the lock pin hole in the clasp head. The lock pin 78 is shorter than the closure pins 72.

The bar 60 in the reamer head has a lock hole 80 within it towards one end thereof. This can be contrasted with the embodiment shown in FIG. 2 in which a lock hole is located centrally on the bar. The shell has a cut out portion 82 in its outer wall.

In order to fasten the reamer head to the clasp head, the collar 70 is retracted along the distal portion 56 to withdraw the closure pins 72 and the lock pin 78 from their respective holes 68, 76 in the clasp head. The bar 60 in the reamer head is then inserted into the openings 66 in the clasp head which communicate with the recesses 64, and twisted relative to the clasp head so that the bar is received firmly within the recesses. Once the bar has been twisted clear of the openings 66, the collar 70 can be released so that it moves outwardly along the distal portion of the retractor, so that the closure pins 72 extend from their respective holes 68 in the clasp head, preventing the bar from inadvertently twisting out of the recesses.

When the lock pin hole 76 is aligned with the axis of the distal portion 56 of the manipulator, the collar can slide fully towards the reamer head so that the lock pin 78 extends through the lock pin hole 76 in the clasp head and into the lock hole 80 in the bar 60. This prevents rotation of the bar (and the reamer head) relative to the clasp and the manipulator. When the lock pin hole 76 is not aligned with the axis of the distal portion of the manipulator, the lock pin 78 is not able to extend from the lock pin hole in the clasp head, and this prevents movement of the collar fully towards the reamer head. However, because the lock pin 78 is shorter than the closure pins 72, the closure pins still serve to prevent the bar 60 from being moved out of the recesses in the clasp head.

The cut out portion 82 in the other wall of the reamer head allows the head to be rotated so that the plane containing the open side of the head is substantially parallel to the axis of the distal portion of the manipulator.

FIGS. 4 to 6 show an assembly of the invention in which the shell component can rotate relative to a clasp on the manipulator, and in which there are a number of defined orientations of the component, for example by means of a plurality of lock holes as in the shell component shown in FIG. 1.

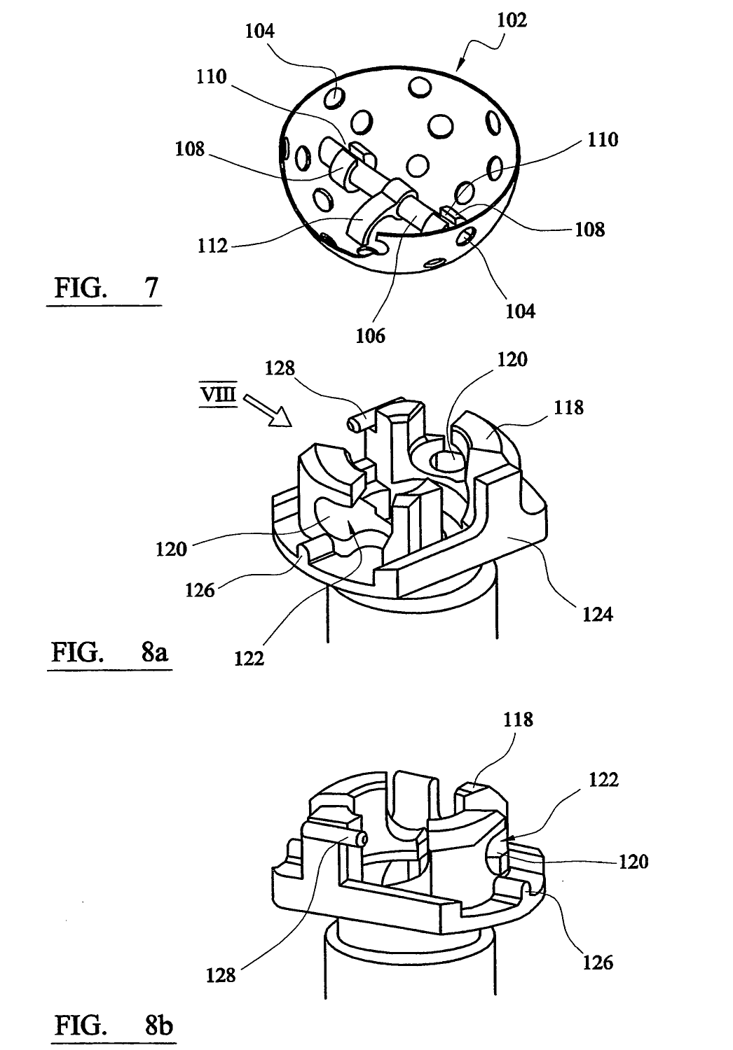

FIG. 4 shows the shell component with the plane defined by the open side of the shell 30 component parallel to the axis 32 of the manipulator 34. FIG. 5 shows the shell component with angle between the plane defined by the open side of the shell component and the axis of the manipulator equal to about 25°. FIG. 6 shows the shell component with angle between the plane defined by the open side of the shell component and the axis of the manipulator equal to about 90°.

It will be common for the reamer head shell component to be deployed for use in the arrangement shown in FIG. 6. However, it is readily apparent from comparison of FIGS. 4 to 6 that the size of the incision that is necessary to locate the reamer in the relevant body cavity is greater when the assembly is in that configuration than when in either of the configurations shown in FIGS. 4 and 5. The present invention makes it possible for the reamer head to be inserted into the body cavity in a small size configuration as shown in FIGS. 4 and 5, and then to be deployed for use in the FIG. 6 configuration once within the cavity.

A cut-out portion in the wall of the shell allows the shell to move from the FIG. 5 configuration to the FIG. 4 configuration, where the handle of the manipulator fits into the cut out portion in the wall of the shell.

FIG. 7 shows a reamer component of an instrument set which comprises a hollow shell 102 with raised cutting teeth 104 on its outer surface. The component has a bar 106 within it, fastened at its ends to the internal surface of the shell, for example by welding. The bar is straight when viewed along the axis of the sphere and also when viewed from one side.

The bar has two collars 108 mounted on it, towards respective opposite ends of the bar. Each of the bars has a groove 110 formed into it. The collar has a circular shape when viewed along the bar and the groove extends radially relative to the circular cross-section of the collar. The groove extends from the surface of the rod in a direction which is parallel to the polar axis of the shell. The rod and its collars can be formed by casting, or from separate parts which are connected together, for example by welding or by means of adhesive, or mechanically etc.

The bar has a hook 112 mounted on it, in the illustrated embodiment at about the midpoint along its length. The hook is turned upwardly towards its free end, generally towards the pole of the shell.

FIGS. 8a and 8b show the clasp assembly of a manipulator which is similar to that shown in FIGS. 2 and 3. The clasp assembly includes a clasp head 118 which has two recesses 120 in it, each of them having an opening 122. The recesses are arranged so that the bar can be retained in the clasp, under two opposites ones of the recesses.

The clasp includes a retaining sleeve 124 which can be moved relative the clasp head 118 along the axis of the clasp. The retaining sleeve has two upwardly facing ridges 126 (of which one only is visible in the FIGS. 8a and 8b). The ridges are aligned with two of the recesses.

The retaining sleeve also presents a hook 128 which is located on the periphery of the sleeve, on a line which extends approximately perpendicular to a line which joins the two ridges 126.

FIG. 9 shows the internal parts of a manipulator in an exploded view, including the clasp head 118 and the retaining sleeve 124. The drawing does not include an external sleeve which can be fitted over the internal parts, for example to allow the instrument to be gripped, and to isolate parts of the instrument which move when the instrument is in use.

The manipulator includes a drive shaft 130 by which rotational drive can be imparted to a reamer or other component fastened to the clasp assembly. The drive shaft can be arranged at an angle to the axis of the component. Drive to the component is transmitted through a universal joint 132 of a conventional kind, which is connected directly to the base 134 of the clamp assembly.

The clamp assembly includes a spring 136 which acts on between retaining sleeve 124 at the upper end of the spring and a collar 138 at its lower end. The collar fits onto the base of the clamp assembly where it is fastened, for example by means of a screw thread or bayonet fixing formations. The spring is therefore able to urge the retaining sleeve upwardly relative to the clamp head, and to allow the retaining sleeve to be displaced reversibly in a downward direction.

The retaining sleeve 124 can be moved downwardly relative to the clasp head. This can be done by grasping the retaining sleeve itself. Alternatively, it can be made to move by remote control, for example by means of an actuator which engages the retaining sleeve. The retaining sleeve has a peripheral groove 140 towards its upper end by which it can engage an actuator for axial movement thereof.

A reamer component (such as that shown in FIG. 7) is connected to the manipulator shown in FIGS. 8 and 9 by locating the bar 106 adjacent to the openings 122 into the recess 120. Rotation of the component head relative to the clasp head causes the bar to be received within the recesses.

The collars 108 on the bar 106 act against the upwardly facing surface of the retaining sleeve 124 on which the ridges 126 are provided. As the reamer component is rotated relative to the clasp head, the collars move across the said upwardly facing surface towards the ridges. Action of the collar against the ridges causes the retaining sleeve to be displaced downwardly against the action of the spring 136 as it is compressed. When the relative rotational positions of the component and the clasp head are such that the ridges are aligned with the grooves 110 in the collars 108, the retaining sleeve moves upwardly so that the ridges are received in the grooves. Further rotation of the reamer component relative to the clasp head is not possible while the ridges are received in the grooves.

As the reamer component is rotated relative to the clasp head, the hook 112 on the bar 106 moves under the hook 128 on the retaining sleeve 124.

FIG. 10a shows a reamer component mounted on the head of the manipulator, with the bar 106 received in the recesses on the clasp head and the grooves on the retaining sleeve received in the grooves in the collars on the bar. The hook 112 on the bar 106 is received under the hook 128 on the retaining sleeve 124. The reamer component is then in its position for use, in which rotational drive can be imparted to it through the drive shaft 130.

The reamer component can be tilted by axial withdrawal of the retaining sleeve 124, as shown in FIG. 10b. Location of the hook 112 on the bar 106 under the hook 128 on the retaining sleeve causes the reamer component to be tilted, about the axis defined by the bar 106 within the recesses on the clasp head.

The reamer component is released from the clasp head by partial axial withdrawal of the retaining sleeve, sufficient to withdraw the ridges on the retaining sleeve from within the grooves on the collars, but not sufficient to cause the hook 112 on the bar 106 to engage the hook 128 on the retaining sleeve and thereby to cause the reamer component to tilt. This then allows the reamer component to be rotated relative to the clasp head so that the bar can be released from within the recesses.

The assembly of the present invention can be used to manipulate other components. For example, it can be used to manipulate instruments other than reamers. It can be used to manipulate implant components, for example the acetabular cup component of a hip joint prosthesis. Generally, the prosthesis will require a suitable mounting for the fastening formation.

1. An assembly for use in orthopaedic surgery, which comprises a component which is to be positioned within a body cavity to engage a bone, the component comprising a hollow shell which is open on one side to allow access to its interior and which has a bar extending across it, and a manipulator having a clasp for engaging the bar so as to fasten the component to the manipulator, in which the clasp allows rotation of the bar so that the angular orientation of the component relative to the manipulator can change.

2. An assembly as claimed in claim 1, in which the clasp has a lock which can engage the bar to restrict rotation of the bar relative to the clasp.

3. An assembly as claimed in claim 2, in which one of the bar and the clasp presents at least one ridge, and the other of the bar and the clasp presents a corresponding groove, arranged so that the ridge can be received in the groove to restrict rotation of the bar relative to the clasp.

4. An assembly as claimed in claim 2, in which the bar has at least one aperture in it, and in which the clasp includes a retractable pin which can be received in the aperture in the bar to lock the bar against rotation relative to the clasp.

5. An assembly as claimed in claim 1, which includes an actuator for causing the angular orientation of the component relative to the manipulator to change.

6. An assembly as claimed in claim 5, in which the actuator comprises an actuator part on the manipulator which can be moved relative to the clasp, and in which the actuator includes a hook which is provided on one of the component and the actuator part, and a recess which is provided in the other of the component and the actuator part, and in which the angular orientation of the component can be changed by moving the actuator part relative to the clasp, which causes the component to move by virtue of the hook being received in the recess.

7. An assembly as claimed in claim 1, in which the shell has a cut out portion towards the open side in a region of its wall that is located approximately opposite to the midpoint of the bar.

8. An assembly as claimed in claim 1, in which the manipulator includes a tube portion and a shaft which can rotate within the tube portion, the clasp being fastened to the shaft so that the component can be rotated relative to the tube portion.

9. An assembly as claimed in claim 1, in which the external surface of shell is symmetrical about an axis of rotation.

10. An assembly as claimed in claim 9, in which the bar intersects the axis of rotational symmetry.

11. An assembly as claimed in claim 9, in which the external surface of the shell defines a part of a sphere.

12. An assembly as claimed in claim 1, in which the portion of the bar which is engaged by the clasp is located within the shell so that, when the component is fastened to the manipulator, the clasp is located at least partially within the shell.

13. An assembly as claimed in claim 12, the external surface of shell is symmetrical about an axis of rotation, and in which the bar intersects the axis of rotational symmetry, the ratio of the distance from where the axis intersects the open side of the shell to the centre of the bar where it intersects the axis, to the length of the axis measured from the open side of the shell to the external surface of the shell opposite the open side being at least about 0.2.

14. An assembly as claimed in claim 1, in which the bar is straight.

15. An assembly as claimed in claim 1, in which the bar is cranked.

16. An assembly as claimed in claim 1, in which the component is a cutting tool, in which the external surface of the shell has cutting teeth.

17. An assembly as claimed in claim 1, in which the component is a component of an orthopaedic joint prosthesis.
